(19) United States
(12) Patent Application Publication
(10) Pub. No.: US 2023/0207071 A1
(43) Pub. Date: Jun. 29, 2023

(54) KNOWLEDGE-GROUNDED COMPLETE CRITERIA GENERATION

(71) Applicant: MICROSOFT TECHNOLOGY LICENSING, LLC, Redmond, WA (US)

(72) Inventors: Tingting ZHAO, Redmond, WA (US); Ke JIANG, Bellevue, WA (US); Liang DU, Redmond, WA (US); Robin ABRAHAM, Redmond, WA (US)

(21) Appl. No.: 17/565,404

(22) Filed: Dec. 29, 2021

(51) Int. Cl. G16H 10/20; G06N 5/04; G06V 30/19

(52) U.S. Cl. CPC G16H 10/20; G06N 5/04; G06V 30/19147; G06V 30/19173

(57) ABSTRACT

Disclosed herein is a model flow that generates eligibility criteria for a clinical trial based on eligibility criteria associated with a protocol title of the trial. Unlike standard black-box generation models, the techniques disclosed herein leverage existing knowledge to enhance the title. The enhanced title also acts as an intermediate between the title and the generated criteria clauses, enabling explicit control of the generated content as well as an explanation of why the generated content is relevant. The resulting workflow is knowledge-grounded, controllable, transparent, and interpretable.

### BACKGROUND

Clinical trials are vital for understanding disease and testing new treatments. However, trial designers face significant challenges recruiting enough participants and establishing optimal patient selection in study populations. Eligibility criteria determine who can participate in a clinical trial. Effectively selecting eligibility criteria—i.e. inclusion criteria and exclusion criteria—is critical to addressing these challenges.

Enrolling optimal patient populations in clinical trials helps provide evidence that the investigational treatment will be safe and effective. Inclusion/exclusion criteria are designed to fulfill this objective. The optimal patient cohort is neither too narrow, such that the applicability is limited, nor too broad, such that the trial cannot demonstrate effectiveness of the treatment. For example, overly restrictive inclusion criteria may limit the applicability or the feasibility of the study. At the same time, a lax exclusion criteria may allow in participants with high risk of an adverse reaction. In addition to the harm caused to participants by adverse reactions, severe adverse reactions may result in a study being canceled. This may delay or even preclude a treatment from coming to market, causing harm to individuals who would have benefited from the treatment. The financial cost of canceling a clinical trial is also significant. For example, having to reset a trial may cost a billion dollars or more.

Existing machine learning techniques are insufficient to generate eligibility criteria from a study's title. For example, generating eligibility criteria based on a study's title may be formulated as a standard sequence-to-sequence (seq2seq) learning problem, where the input sequence is the title and the output sequence is the eligibility criteria. However, there are many drawbacks to this approach. For example, the generated eligibility criteria mostly inherit information from the title, lacking a richness found in hand-written criteria. Furthermore, the criteria section of a study protocol is usually very long—beyond the maximal sequence length of commonly used transformer-based models. Another drawback is that, unlike typical documents in which sentences have a natural order, a study's eligibility criteria are not restricted to a particular order, which hinders model training. Finally, there is little control as to what criteria will be generated, and no way to assess how much the model has learned.

It is with respect to these technical issues and others that the present disclosure is made.

### SUMMARY

Disclosed herein is a model flow that generates eligibility criteria for a clinical trial based on a protocol title of the trial. Unlike standard black-box generation models, the techniques disclosed herein leverage existing knowledge to enhance the title. The enhanced title also acts as an intermediate between the title and the generated criteria clauses, enabling explicit control of the generated content as well as an explanation of why the generated content is relevant. The resulting workflow is knowledge-grounded, controllable, transparent, and interpretable.

In some configurations, a plurality of clinical trial protocol titles and associated eligibility criteria are received. The protocol titles and associated eligibility criteria are used to train an external knowledge machine learning model. The external knowledge machine learning model may then be used to identify external knowledge associated with some or all of the protocol titles. As referred to herein, external knowledge refers to context information, related information, or any other information that is associated with at least a portion of a protocol title.

In some configurations, an eligibility criteria machine learning model is trained using the protocol titles, the external knowledge associated with the protocol titles, and the associated eligibility criteria. The eligibility criteria machine learning model may then be used on a particular protocol title of a particular clinical trial to generate eligibility criteria for that particular clinical trial. In some configurations, a clinical trial designer may iteratively generate eligibility criteria by modifying the particular protocol title. Additionally, or alternatively, the clinical trial designer may affect which eligibility criteria are generated by modifying some or all of the external knowledge manually.

Features and technical benefits other than those explicitly described above will be apparent from a reading of the following Detailed Description and a review of the associated drawings. This Summary is provided to introduce a selection of concepts in a simplified form that are further described below in the Detailed Description. This Summary is not intended to identify key or essential features of the claimed subject matter, nor is it intended to be used as an aid in determining the scope of the claimed subject matter. The term “techniques,” for instance, may refer to system(s), method(s), computer-readable instructions, module(s), algorithms, hardware logic, and/or operation(s) as permitted by the context described above and throughout the document.

### BRIEF DESCRIPTION OF THE DRAWINGS

The Detailed Description is described with reference to the accompanying figures. In the figures, the left-most digit(s) of a reference number identifies the figure in which the reference number first appears. The same reference numbers in different figures indicate similar or identical items. References made to individual items of a plurality of items can use a reference number with a letter of a sequence of letters to refer to each individual item. Generic references to the items may use the specific reference number without the sequence of letters.

FIG. 1 illustrates a model flow used to generate eligibility criteria for a clinical trial.

FIG. 2 illustrates training one or more external knowledge models usable to identify external knowledge associated with a protocol title of a clinical trial.

FIG. 3 illustrates using external knowledge models to identify external knowledge for each of a corpus of protocol titles.

FIG. 4 illustrates training a criteria model based on a corpus of protocol titles that has been enhanced by external knowledge.

FIG. 5 illustrates identifying one or more entities from a protocol title of a clinical trial using an information retrieval technique.

FIG. 6 illustrates generating eligibility criteria from a protocol title of a clinical trial using a sequence-to-sequence technique.

FIG. 7 is a flow diagram showing aspects of a routine for the disclosed techniques.

FIG. 8 is a computer architecture diagram illustrating an illustrative computer hardware and software architecture for a computing system capable of implementing aspects of the techniques and technologies presented herein.

### DETAILED DESCRIPTION

Disclosed herein is a model flow that generates eligibility criteria for a clinical trial based on a protocol title of the trial. Unlike standard black-box generation models, the techniques disclosed herein leverage existing knowledge to enhance the title. The enhanced title also acts as an intermediate between the title and the generated criteria clauses, enabling explicit control of the generated content as well as an explanation of why the generated content is relevant. The resulting workflow is knowledge-grounded, controllable, transparent, and interpretable.

As discussed above, existing techniques to train a machine learning model to generate eligibility criteria from a protocol title have proved insufficient. Table 1 shows an example of the standard seq2seq generation results. It shows that the generated clauses mostly inherit the information from the title without adding new knowledge. Also, the generated clauses have very similar linguistic patterns.

TABLE 1
Generated example from the standard seq2seq model

Title: Basic and Clinical Studies in Reinforcing Positive Behaviors in
Intellectual and Developmental Disabilities
Generated Inclusion Criteria:

Patients with cognitive impairments
Patients older than 20 years old
Patients with intellectual and developmental disabilities
Patients with developmental disabilities who are able to
walk independently (to avoid injury)
Patients who are able to understand written and spoken French

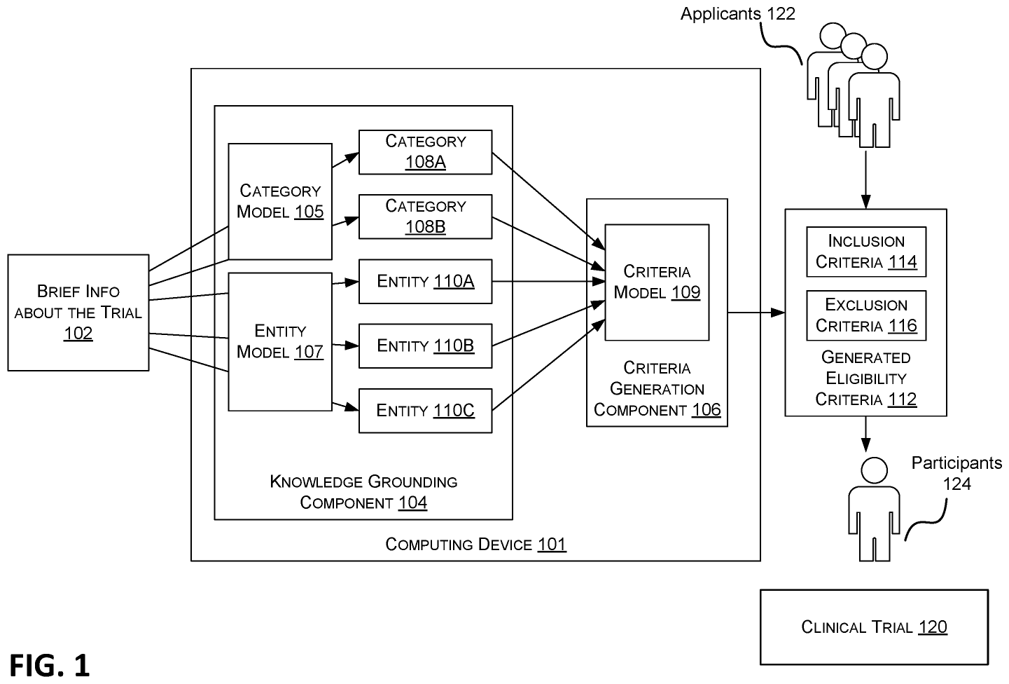

FIG. 1 illustrates a model flow used to generate eligibility criteria 112 for a clinical trial 120. Clinical trials are experiments or observations done in clinical research. Such prospective biomedical or behavioral research studies on human participants are designed to answer specific questions about biomedical or behavioral interventions, including new treatments such as novel vaccines, drugs, dietary choices, dietary supplements, and medical devices. Clinical trials are also referred to as studies interchangeably throughout this document.

As illustrated, eligibility criteria 112 may refer to one of at least two types of criteria: inclusion criteria 114, exclusion criteria 116, or a combination thereof. Eligibility criteria 112 determines which applicants 122 may become participants 124 in clinical trial 120. Specifically, inclusion criteria 114 sets out one or more criterion that an applicant 122 must meet before being admitted as a participant 124 of clinical trial 120. However, even if an applicant 122 meets all of the inclusion criteria 114, that applicant 122 will be excluded from clinical trial 120 if they meet one or more of exclusion criteria 116. As discussed briefly above, inclusion criteria 114 and exclusion criteria 116 are crucial to designing clinical trial 120 such that enough participants may be recruited to allow for a meaningful result while avoiding participants that may be harmed.

As illustrated, computing device 101 receives brief information 102 about clinical trial 120. In some configurations, brief information 102 about clinical trial 120 is a protocol title, also referred to as a title, of clinical trial 120. A title of a clinical trial 120 typically describes the trial in succinct terms, calling attention to key aspects. One example of a title of a clinical trial is “Basic and Clinical Studies in Reinforcing Positive Behaviors in Intellectual and Developmental Disabilities.”

Instead of providing brief information 102 directly to a criteria generation component, the knowledge grounding component 104 of the model flow first enhances the brief information 102 by identifying external knowledge associated with brief information 102. Enhancing the brief information 102 with external knowledge enables richer, more accurate, and a greater variety of eligibility criteria 112 to be generated.

External knowledge refers to any information, data, or other knowledge associated with brief information 102 or a portion thereof. Different types of external knowledge are contemplated, and may be used to enhance brief information 102 alone or in combination. Two examples of external knowledge are categories and entities, although other types of external knowledge are similarly contemplated. In some configurations, a category refers to a classification of eligibility criteria associated with the brief information 102 as a whole. An entity, in contrast, refers to a noun phrase, a clause, or some other sub-section of the eligibility criteria associated with the brief information 102.

Adding external knowledge to brief information 102 may expand the scope of subject matter included in the generated eligibility criteria. For example, external knowledge may introduce related concepts that are not listed in the brief information 102 itself. At the same time, adding external knowledge to brief information 102 may constrain the subject matter addressed by the generated eligibility criteria. For example, the added external knowledge might constrain the generation of the eligibility criteria by guiding the generation to be related to the added knowledge. In this way, generation of eligibility criteria may be controlled by selecting which external knowledge is made available when training a criteria generation model.

As illustrated, knowledge grounding component 104 uses category model 105 to identify categories 108 of eligibility criteria associated with brief information 102. In some configurations, category model 105 is a machine learning model trained on a corpus of study titles and categories associated with the corresponding eligibility criteria. That is, the input when training model 105 is a study title, and the output is all of the categories associated the eligibility criteria that are associated with the study title. Clinical trial protocol titles and the associated eligibility criteria may be obtained from websites that register and manage clinical trials, such as a clinical trials website maintained by the national institutes of health. When generating eligibility criteria for a particular study title, category model 105 may be used to infer categories for the particular study title. However, any other technique for associating a study title with one or more of a defined set of categories is similarly contemplated.

One example of a procedure for training category model 105 begins by collecting the title and eligibility criteria of previously published clinical trials. Then, at step two, for each clause of the eligibility criteria, a category is identified. For example, a subject matter expert may review the eligibility criteria and identify one or more categories associated with each clause. Once categories of the eligibility criteria have been identified, step three combines and de-duplicates the categories, yielding the ground truth output used to train category model 105. Finally, at step four, a multi-label classification model is trained with the title as the input and the categories from step three as the output. In some configurations, these steps are performed for inclusion criteria and exclusion criteria separately.

For example, the title “A Phase I Study Combining NeoVax, a Personalized NeoAntigen Cancer Vaccine, With Ipilimumab to Treat High-risk Renal Cell Carcinoma” may be one of a corpus of study titles. The study may have eligibility criteria, such as “Age≥18 years”. A subject matter expert may label the eligibility criteria with one of a defined set of categories. For this example, the eligibility criteria may be labeled with the category “age”. Another example of an eligibility criteria from the same study is “Patients should have suspected stage III or stage IV clear cell renal cell carcinoma (ccRCC), with anticipation that all disease can be surgically resected. Confirmation of clear cell histology, final stage (III or IV), and removal of all disease will be done after the surgery, and will be required for further participation of the trial”, which an expert may label with the category “Diagnostic”.

As mentioned briefly above, the categories used to label eligibility criteria may be selected from a predefined set of categories. The specific categories that are available may vary according to the goals of the study designers. For example, if “Age” is included as one of the possible categories, then the generated eligibility criteria may be more sensitive to age-related terms in the study title.

Once category model 105 has been trained it may be used to infer one or more categories for a particular study title. In some configurations, the study title is provided by a study designer in the process of generating eligibility criteria. For example, category model 105 may infer categories 108 of “Age” and “Diagnostic” from the study title “Basic and Clinical Studies in Reinforcing Positive Behaviors in Intellectual and Developmental Disabilities”. In some configurations, category model 105 infers categories based on the study title as a whole.

Entity model 107 identifies entities 110 associated with brief information 102. Similar to category model 105, entity model 107 may be a machine learning model trained based on a corpus of study titles and entities associated with corresponding eligibility criteria. For example, the same eligibility criteria “Patients should have suspected stage III or stage IV clear cell renal cell carcinoma (ccRCC), with anticipation that all disease can be surgically resected. Confirmation of clear cell histology, final stage (III or IV), and removal of all disease will be done after the surgery, and will be required for further participation of the trial” may be labeled by a subject matter expert as having the entity “Renal Cell Carcinoma.” “Renal Cell Carcinoma” may be one of a predefined set of entities, or may be extracted directly from the eligibility criteria.

One example of a procedure for training entity model 107 mirrors the procedure described above for training category model 105. First, the title and eligibility criteria of previously published clinical trials is collected. Then, at step two, for each clause of the eligibility criteria, an entity is identified. For example, a subject matter expert may review the eligibility criteria and identify one or more entities associated with each clause. Once entities of the eligibility criteria have been identified, step three combines and de-duplicates the entities, yielding the ground truth output used to train entity model 107. Finally, at step four, a multi-label classification model is trained with the title as the input and the entities from step three as the output. In some configurations, these steps are performed for inclusion criteria and exclusion criteria separately when training entity model 107.

In some configurations, particularly when the subject matter of the eligibility criteria is technical, different words or phrases may be used to refer to the same concept. The effectiveness of the trained models may be degraded due to these differences in entity phrasing and word choice. In order to address this issue, knowledge grounding component 104 may apply an ontology component to normalize the entities 110 used to train entity model 107. In the case of clinical trials, a medical ontology canonicalizes medical terms and acronyms so that different phrases with the same meaning are represented using the same entity. In some configurations, canonicalizing terminology enables entity model 107 to be trained using a classification technique in which each normalized entity is treated as a class.

Once entity model 107 has been trained it may be used to infer one or more entities for a particular study title. As with category model 105, or any other model used to enhance a study title, the study title may be provided by a study designer in the process of generating eligibility criteria. Examples of entities 110 inferred from entity model 107 include “Behavior Therapy” and “Intellectual Disability.” For example, “Behavior Therapy” may have been associated with the phrase “behavioral treatment” in the criteria “children currently receiving intensive (i.e., 15 or more hours per week), function-based, behavioral treatment for their problem behavior through the school or another program”. “Intellectual Disability” may have been associated with the phrase “intellectual disability” in the criteria “IQ and adaptive behavior scores between 35 and 70 (i.e., mild to moderate intellectual disability)”.

In some configurations, criteria model 109 of criteria generation component 106 is a machine learning model trained to generate eligibility criteria 112. While training criteria model 109, study titles 102 and external knowledge such as identified categories 108 are provided as inputs and the corresponding eligibility criteria are provided as outputs. For example, if the category “Age” was inferred by category model 105 from the eligibility criteria “Age≥18 years”, then the study title 102 and the category “Age” would be an input and the eligibility criteria “Age≥18 years” would be an output. As illustrated, the model flow implemented by computing device 101 trains criteria model 109 with the brief information 102 and one or more types of external knowledge such as the identified categories 108 or the identified entities 110. In some configurations, criteria model 109 is trained with brief information 102 that has been enhanced with a single type of external knowledge, e.g. with identified entities 110 but not identified categories 108.

Once trained, criteria model 109 may infer eligibility criteria 112, including inclusion criteria 114 and exclusion criteria 116. In some configurations, for a particular study title, external knowledge is obtained, e.g. an entity 110 is obtained from entity model 107 as discussed above. For each piece of external knowledge, e.g. for each entity 110 obtained, the particular study title and the entity 110 are provided to criteria model 109 to infer one or more eligibility criteria. In some configurations, each piece of external knowledge is used in combination with the particular study title to infer a single eligibility criterion. FIG. 1 illustrates a single computing device 101 both training and performing inference with models 105, 107, and 109, but this is just one embodiment, and it is similarly contemplated that multiple computing devices may be used to train or perform inference with one or more of models 105, 107, and 109.

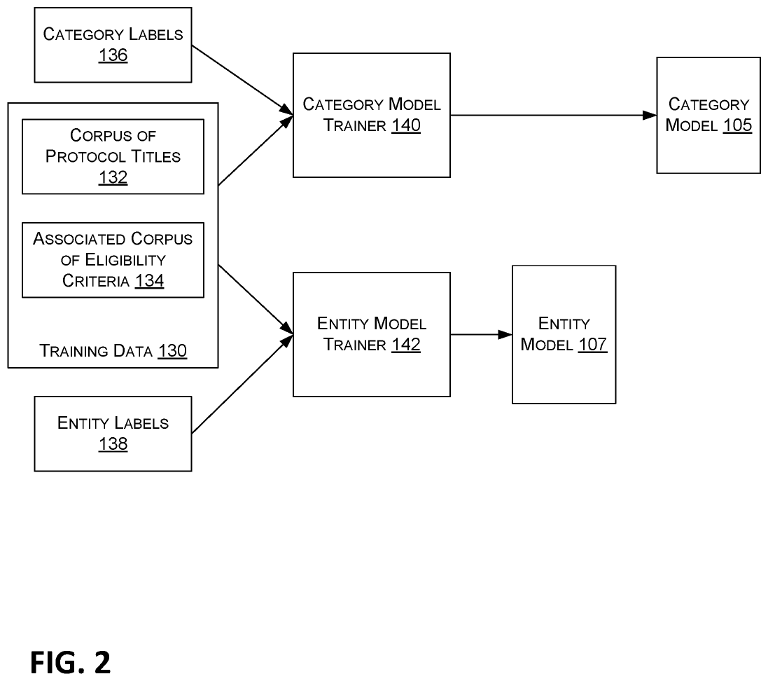

FIGS. 2-4 illustrate a process for training a criteria model 109 to generate eligibility criteria 112 for clinical trial 120 from a particular brief information 102 of clinical trial 120. The process illustrated in FIGS. 2-4 may be implemented on computing device 101.

FIG. 2 illustrates training one or more external knowledge models usable to identify external knowledge associated with a protocol title of a clinical trial. In some configurations, training data 130 includes a corpus of protocol titles 132 and an associated corpus of eligibility criteria 134. Each of eligibility criteria 134 may indicate whether that eligibility criteria is an inclusion criteria or an exclusion criteria. For example, one of the corpus of protocol titles 132 may be associated with a subset of the associated corpus of eligibility criteria 134 because a previously administered clinical trial was published with that title one title and having the subset of eligibility criteria. As mentioned above, this information may be downloaded in bulk from websites that register and manage clinical trials.

If the external knowledge type used to enhance study titles is “category”, then training data 130 is provided to category model trainer 140. A subject matter expert may label some or all of the associated corpus of eligibility criteria 134 with one of a predefined set of categories, which are also provided to category model trainer 140. Category model trainer 140 may use protocol titles 132 as inputs and the category labels 136 of the associated eligibility criteria 134 as outputs to train a machine learning model as category model 105. In some configurations, category model trainer 140 also provides whether an eligibility criteria is an “inclusion criteria” or “exclusion criteria” as input when training category model 105.

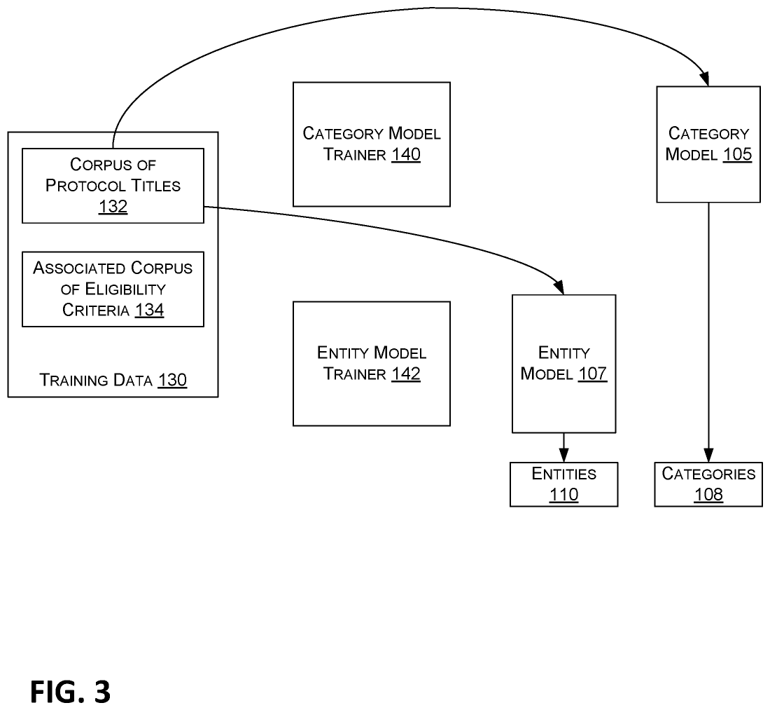

If one of the external knowledge types used to enhance study titles is “entity”, then training data 130 is provided to entity model trainer 142. A subject matter expert may label some or all of the associated corpus of eligibility criteria 134 with one or some of a predefined set of entities, which are also provided to entity model trainer 142. Training data 130 may additionally include an indication of which eligibility criteria are inclusion criteria 114 and which eligibility criteria are exclusion criteria 116. Entity model trainer 142 may use one or more of a number of techniques that are discussed in more detail below in conjunction with FIGS. 3-5 to train entity model 107. Briefly, entity model trainer 142 uses protocol titles 132 as inputs and entity labels 138 of the associated eligibility criteria 134 as outputs to train entity model 107.

FIG. 3 illustrates using external knowledge models 105 and 107 to identify external knowledge for each of a corpus of protocol titles 132. In some configurations, the corpus of protocol titles is enhanced with external knowledge provided by category model 105, entity model 107, and/or any other sources of external knowledge. As discussed above in conjunction with FIG. 1, one or more of entities 110 or categories 108 are external knowledge associated with each of the protocol titles 132, depending on whether knowledge grounding component is configured to augment protocol titles with categories, entities, or some other type of external knowledge.

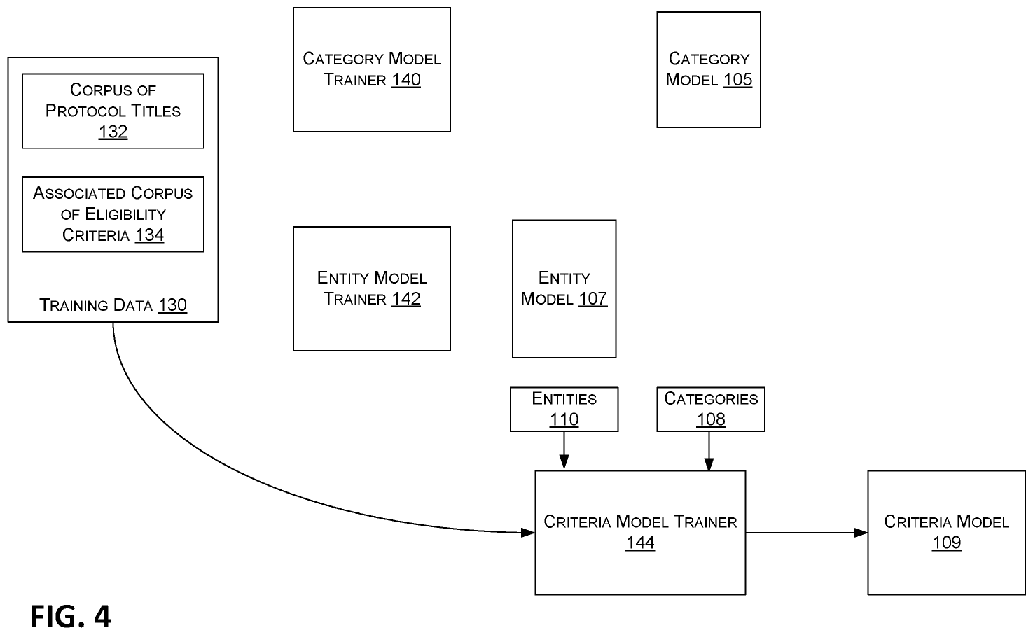

FIG. 4 illustrates training criteria model 109 based on the corpus of protocol titles 132 that has been enhanced by external knowledge—e.g. entities 110 and/or categories 108. As illustrated, training data 130, including protocol titles 132 and associated eligibility criteria 134, are provided to criteria model trainer 144. Additionally, entities 110 and/or categories 108 are also provided to criteria model trainer 144. While FIG. 4 illustrates using two types of external knowledge (entities alone, categories alone, another type of external knowledge alone, or some combination thereof), more, fewer, different, and additional types of external knowledge are similarly contemplated. When a piece of external knowledge is associated with a protocol title, the protocol title may be referred to as an enhanced protocol title.

Criteria model trainer 144 may then be used to train a machine learning model, referred to as criteria model 109. The enhanced protocol titles 132, e.g. protocol titles 132 in association with corresponding entities 110 and/or categories 108, may be used as input while the associated eligibility criteria 134 may be used as output while training criteria model 109.

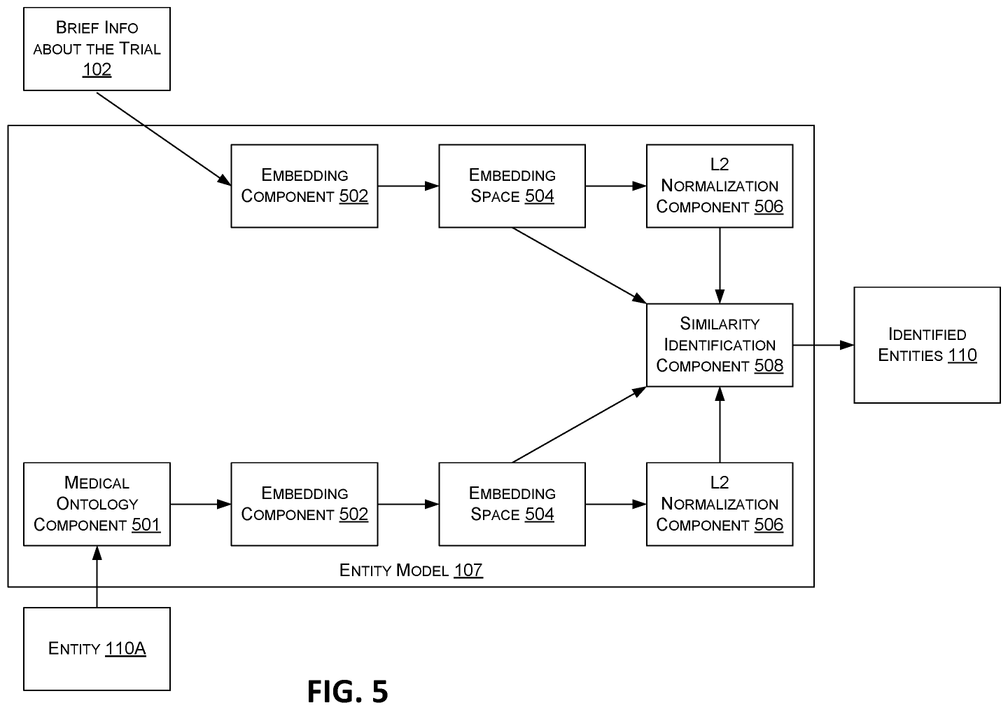

FIG. 5 illustrates identifying one or more entities 110 from a protocol title 102 of a clinical trial 120 using an information retrieval technique. As illustrated, brief info 102 about the trial 120 and entity 110A are separately provided to entity model 107 of knowledge grounding component 104. With the information retrieval technique, the brief info 102 is analogous to a search query, and the entities 110 are analogous to a set of documents being searched. Finding the best search N search results therefore identifies the N entities 110 most likely associated with the brief information 102. FIG. 5 illustrates one comparison of brief info 102 to one of entities 110. The results of these comparisons may then be ordered based on the similarity between the brief info 102 and each entity 110. A pre-defined number of the most similar entities may then be used as inputs to eligibility criteria model 109 when generating eligibility criteria 112.

As illustrated, a single evaluation of an entity 110A begins by processing entity 110A with an ontology, such as medical ontology 501. Brief info 102 and normalized entity 110A may then be provided to embedding component 502, which transforms the brief info 102 and entity 110A into embedding space 504—i.e. into same-length vectors. Entity model 107 may then perform an L2 normalization 506—i.e. normalizing the embedding vectors of embedding space 504 into vectors with unit length. The results of L2 normalization 506 may then be provided to similarity identification component 508, which generates a numeric similarity score for brief info 102 and entity 110A. In some embodiments, the numeric similarity score for brief info 102 and entity 110A is found by computing an inner product of the normalized embedding vectors associated with brief info 102 and entity 110A. Computing the inner product of the normalized embedding vectors generates a cosine similarity between brief info 102 and entity 110A. Entity model 107 then orders the similarity scores for each of entities 110 and selects the N most similar entities as selected entities 110.

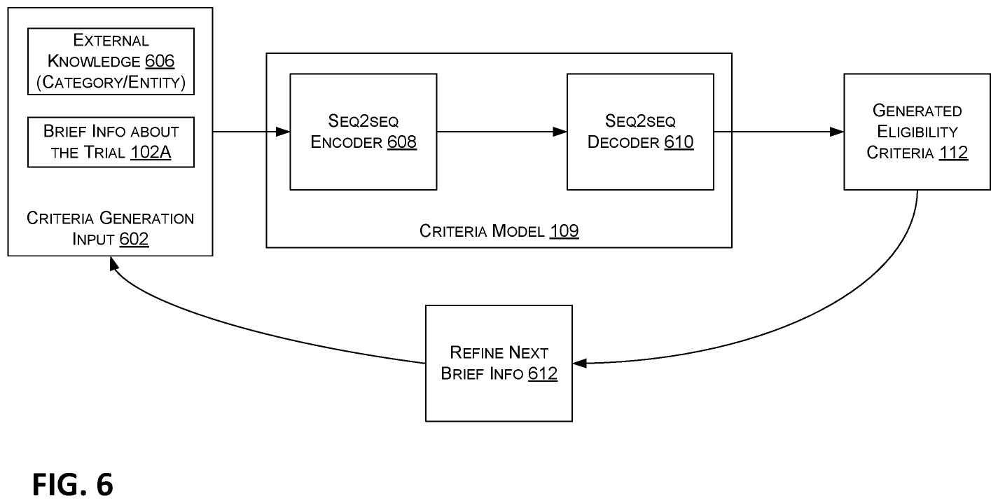

FIG. 6 illustrates generating eligibility criteria 112 from a protocol title 102 of a clinical trial 120 using a sequence-to-sequence technique. In some configurations, the operations of FIG. 6 are performed by criteria generation component 106. As illustrated, criteria generation input 602 is provided to criteria model 109, which infers generated eligibility criteria 112.

Criteria generation input 602 may include a specific brief info 102A. For example, a clinical trial designer may provide brief info 102A while designing clinical trial 120. In some configurations, the clinical trial designer may iteratively refine 612 the brief information 102A, e.g. by submitting brief information 102B, 102C, etc. Once the generated eligibility criteria 112 has been generated for a particular brief information 102, the clinical trial designer may take this information in consideration when drafting the next brief info 102.

Criteria generation input 602 may also specify a number of criteria to generate. Criteria generation input 602 may also include external knowledge 606. External knowledge may be identified from brief info 102A by using the techniques described in FIGS. 2-4. As illustrated, criteria generation input 602 may be used as input to the sequence-to-sequence encoder 608, while the eligibility criteria are output from the sequence-to-sequence decoder 610.

In some configurations, in addition to or as an alternative to modifying the brief info 102A, external knowledge may be altered, deleted, augmented, replaced, or otherwise modified by a clinical trial designer between iterations. These modifications will affect the eligibility criteria generated by criteria model 109. Modifying this intermediate data allows users of the disclosed embodiments an additional tool to control the generated eligibility criteria. In some configurations, the external knowledge associated with a particular brief information 102 gives a clinical trial designer insight into why eligibility criteria are being generated.

An example of eligibility criteria generated by the disclosed embodiments is presented below in TABLE 2. In this example, entities 110 were used to enhance the brief info 102. The entities 110 that were identified are listed at the beginning of each criterion. In this example, the same protocol title 102 is the same as was used in the example depicted above in TABLE 1. However, due to the use of external knowledge, the generated criteria are more diverse and cover different aspects of “intellectual and developmental disabilities”, rather than focusing on the exact terms. This not only gives the trial designer a sense of control but also opens the possibility of the feedback loop discussed above.

TABLE 2
Generated example utilizing external knowledge

Title: Basic and Clinical Studies in Reinforcing Positive Behaviors in
Intellectual and Developmental Disabilities
Generated Inclusion Criteria:

Behavior Therapy: Treatment with permitted medications (at a
stable dose for 12 weeks before screening) and behavioral
therapy regimens (regimens stable for 6 weeks before screening),
with the intent that such treatments remain stable throughout
the study and with no expected changes before the Week 24 visit
Intellectual Disability: Clinical diagnosis of syndromic or
isolated severe intellectual disability (IQ 50) without a
molecular diagnosis
Abnormal behavior: Participants must report some impairment
in daily functioning as a result of emotional or behavior problems
based on a series of questions adapted from the WHODAS
Pervasive Development Disorder: pervasive developmental disorder
Developmentally delayed: Developmentally delayed with Mullen
Scales of Early Learning composite score below 85 (1
Standard Deviation below the mean)
Emotional Control: Specific inclusion criteria for emotional control
group
Cognitive training: 10 hours of previous cognitive training

Generated Exclusion Criteria:

Developmental Disabilities: Developmental disability or cognitive
impairment that in the opinion of the investigator would preclude
adequate comprehension of the consent form and/or ability to
record study measurements
Child attention deficit disorder: Children with ADD/ADHD,
autism or Down's syndrome and children with a history of
behavioral issues that required previous management
Neurodevelopmental Disorders: OCD patients - comorbidity with
neurodevelopmental disorders (autism, mental retardation),
current psychotic disorders, current substance dependence or abuse,
bipolar mood disorder according to evaluation using semi structured
interview for DSM IV diagnoses (SCID I)
Autistic Disorder: Symptoms better explained by axis 2 diagnosis
(e.g. autism or borderline personality disorder)
Intellectual Disability: Intellectual disability/active mental
illness or active substance abuse
Expressive language difficulties: Difficulty in language expression

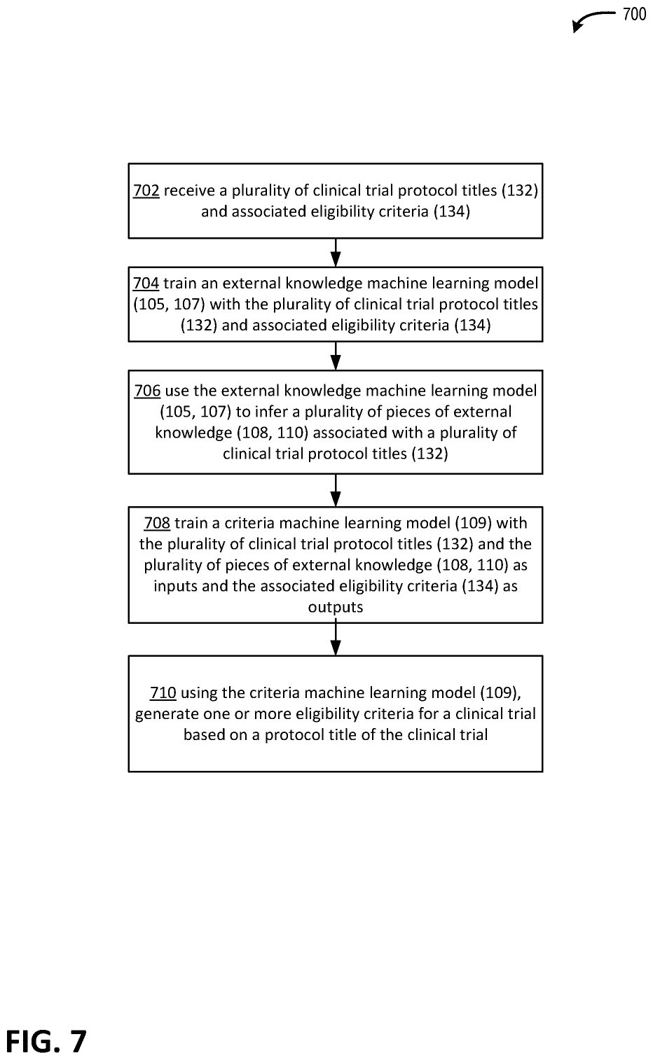

FIG. 7 is a flow diagram showing aspects of a routine for the disclosed techniques. Routine 700 begins at step 702, where a plurality of clinical trial protocol titles 132 and associated eligibility criteria 134 are received by computing device 101. The clinical trial protocol titles 132 and associated eligibility criteria 134 may be historical titles and eligibility criteria used for previously conducted clinical trials.

Routine 700 then proceeds to step 704, where an external knowledge machine learning model 105, 107 is trained with the plurality of clinical trial protocol titles 132 and associated eligibility criteria 134.

The routine then proceeds to step 706, where the external knowledge machine learning model 105, 107 is used to infer a plurality of pieces of external knowledge 108, 110 associated with a plurality of clinical trial protocol titles 132.

The routine then proceeds to step 708, where a criteria machine learning model 109 is trained with the plurality of clinical trial protocol titles 132 and the plurality of pieces of external knowledge 108, 110 as inputs and the associated eligibility criteria 134 as outputs.

The routine then proceeds to step 710, where the criteria machine learning model 109 is used to generate one or more eligibility criteria 112 for a particular clinical 120 trial based on a protocol title 102 of the particular clinical trial 120 that has been enhanced with external knowledge.

It should be understood that the operations of the methods disclosed herein are not necessarily presented in any particular order and that performance of some or all of the operations in an alternative order(s) is possible and is contemplated. The operations have been presented in the demonstrated order for ease of description and illustration. Operations may be added, omitted, and/or performed simultaneously, without departing from the scope of the appended claims.

It also should be understood that the illustrated methods can end at any time and need not be performed in its entirety. Some or all operations of the methods, and/or substantially equivalent operations, can be performed by execution of computer-readable instructions included on a computer-storage media and computer-readable media, as defined herein. The term “computer-readable instructions,” and variants thereof, as used in the description and claims, is used expansively herein to include routines, applications, application modules, program modules, programs, components, data structures, algorithms, and the like. Computer-readable instructions can be implemented on various system configurations, including single-processor or multiprocessor systems, minicomputers, mainframe computers, personal computers, hand-held computing devices, microprocessor-based, programmable consumer electronics, combinations thereof, and the like.

Thus, it should be appreciated that the logical operations described herein are implemented (1) as a sequence of computer implemented acts or program modules running on a computing system and/or (2) as interconnected machine logic circuits or circuit modules within the computing system. The implementation is a matter of choice dependent on the performance and other requirements of the computing system. Accordingly, the logical operations described herein are referred to variously as states, operations, structural devices, acts, or modules. These operations, structural devices, acts, and modules may be implemented in software, in firmware, in special purpose digital logic, and any combination thereof.

Although FIG. 7 refers to the components depicted in the present application, it can be appreciated that the operations of the routine 700 may be also implemented in many other ways. For example, the routine 700 may be implemented, at least in part, by a processor of another remote computer or a local circuit. In addition, one or more of the operations of the routine 700 may alternatively or additionally be implemented, at least in part, by a chipset working alone or in conjunction with other software modules.

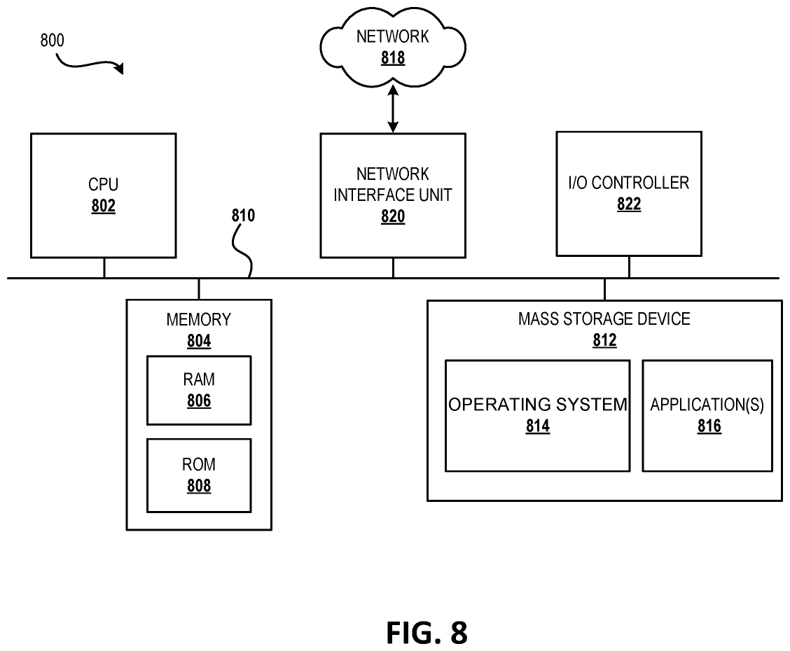

FIG. 8 shows additional details of an example computer architecture 800 for a device, such as computing device 101, capable of executing computer instructions (e.g., a module or a program component described herein). The computer architecture 800 illustrated in FIG. 8 includes processing unit(s) 802, a system memory 804, including a random-access memory 806 (“RAM”) and a read-only memory (“ROM”) 808, and a system bus 810 that couples the memory 804 to the processing unit(s) 802.

Processing unit(s), such as processing unit(s) 802, can represent, for example, a CPU-type processing unit, a GPU-type processing unit, a field-programmable gate array (FPGA), another class of digital signal processor (DSP), or other hardware logic components that may, in some instances, be driven by a CPU. For example, and without limitation, illustrative types of hardware logic components that can be used include Application-Specific Integrated Circuits (ASICs), Application-Specific Standard Products (ASSPs), System-on-a-Chip Systems (SOCs), Complex Programmable Logic Devices (CPLDs), etc.

A basic input/output system containing the basic routines that help to transfer information between elements within the computer architecture 800, such as during startup, is stored in the ROM 808. The computer architecture 800 further includes a mass storage device 812 for storing an operating system 814, application(s) 816 (e.g., criteria model trainer 144), and other data described herein.

The mass storage device 812 is connected to processing unit(s) 802 through a mass storage controller connected to the bus 810. The mass storage device 812 and its associated computer-readable media provide non-volatile storage for the computer architecture 800. Although the description of computer-readable media contained herein refers to a mass storage device, it should be appreciated by those skilled in the art that computer-readable media can be any available computer-readable storage media or communication media that can be accessed by the computer architecture 800.

Computer-readable media can include computer-readable storage media and/or communication media. Computer-readable storage media can include one or more of volatile memory, nonvolatile memory, and/or other persistent and/or auxiliary computer storage media, removable and non-removable computer storage media implemented in any method or technology for storage of information such as computer-readable instructions, data structures, program modules, or other data. Thus, computer storage media includes tangible and/or physical forms of media included in a device and/or hardware component that is part of a device or external to a device, including but not limited to random access memory (RAM), static random-access memory (SRAM), dynamic random-access memory (DRAM), phase change memory (PCM), read-only memory (ROM), erasable programmable read-only memory (EPROM), electrically erasable programmable read-only memory (EEPROM), flash memory, compact disc read-only memory (CD-ROM), digital versatile disks (DVDs), optical cards or other optical storage media, magnetic cassettes, magnetic tape, magnetic disk storage, magnetic cards or other magnetic storage devices or media, solid-state memory devices, storage arrays, network attached storage, storage area networks, hosted computer storage or any other storage memory, storage device, and/or storage medium that can be used to store and maintain information for access by a computing device.

In contrast to computer-readable storage media, communication media can embody computer-readable instructions, data structures, program modules, or other data in a modulated data signal, such as a carrier wave, or other transmission mechanism. As defined herein, computer storage media does not include communication media. That is, computer-readable storage media does not include communications media consisting solely of a modulated data signal, a carrier wave, or a propagated signal, per se.

According to various configurations, the computer architecture 800 may operate in a networked environment using logical connections to remote computers through the network 818. The computer architecture 800 may connect to the network 818 through a network interface unit 820 connected to the bus 810. The computer architecture 800 also may include an input/output controller 822 for receiving and processing input from a number of other devices, including a keyboard, mouse, touch, or electronic stylus or pen. Similarly, the input/output controller 822 may provide output to a display screen, speaker, or other type of output device.

It should be appreciated that the software components described herein may, when loaded into the processing unit(s) 802 and executed, transform the processing unit(s) 802 and the overall computer architecture 800 from a general-purpose computing system into a special-purpose computing system customized to facilitate the functionality presented herein. The processing unit(s) 802 may be constructed from any number of transistors or other discrete circuit elements, which may individually or collectively assume any number of states. More specifically, the processing unit(s) 802 may operate as a finite-state machine, in response to executable instructions contained within the software modules disclosed herein. These computer-executable instructions may transform the processing unit(s) 802 by specifying how the processing unit(s) 802 transition between states, thereby transforming the transistors or other discrete hardware elements constituting the processing unit(s) 802.

The present disclosure is supplemented by the following example clauses.

Example 1: A method for generating eligibility criteria, comprising: receiving a brief description of a clinical trial; determining external knowledge derived from the brief description of the clinical trial; using a machine learning model to infer, based on the brief description and the selected external knowledge, eligibility criteria for the clinical trial.

Example 2: The method of Example 1, wherein the external knowledge includes a category of an eligibility criteria associated with the brief description.

Example 3: The method of Example 2, wherein the category is inferred from a machine learning model trained on a corpus of clinical trial eligibility criteria that has been labeled with semantic categories.

Example 4: The method of Example 1, wherein the external knowledge includes an entity associated with a portion of an eligibility criteria associated with the brief description of the clinical trial.

Example 5: The method of Example 4, wherein the entity is identified using a machine learning model that utilizes extreme multi-label classification to associate the portion of the eligibility criteria associated with the brief description with one of a number of entities.

Example 6: The method of Example 4, wherein the entity is selected using a sequence-to-sequence technique with the brief description of the clinical trial as an input and the entity as an output.

Example 7: The method of Example 4, wherein entity selection is framed as an information retrieval problem wherein the brief description comprises a query and entities comprise documents that are searched.

Example 8: The method of Example 1, wherein the brief description of the clinical trial comprises a protocol title of the clinical trial.

Example 9: A device comprising: one or more processors; and a computer-readable storage medium having encoded thereon computer-executable instructions that cause the one or more processors to: receive a plurality of clinical trial protocol titles and associated eligibility criteria; train an external knowledge machine learning model with the plurality of clinical trial protocol titles and associated eligibility criteria, wherein the external knowledge machine learning model identifies external knowledge associated with a portion of one of the associated eligibility criteria; use the external knowledge machine learning model to infer a plurality of pieces of external knowledge associated with a plurality of clinical trial protocol titles; train a criteria machine learning model with the plurality of clinical trial protocol titles and the plurality of pieces of external knowledge as inputs and the associated eligibility criteria as outputs; and using the criteria machine learning model, generate one or more eligibility criteria for a clinical trial based on a protocol title of the clinical trial.

Example 10: The device of Example 9, wherein the instructions further cause the one or more processors to: using the external knowledge machine learning model, identify a plurality of entities associated with eligibility criteria associated with the protocol title of the clinical trial, wherein training the criteria machine learning model is based in part on the identified plurality of entities.

Example 11: The device of Example 9, wherein the eligibility criteria includes an inclusion criteria or an exclusion criteria.

Example 12: The device of Example 9, wherein the instructions further cause the one or more processors to: normalize medical terminology within the eligibility criteria associated with the protocol title using a medical ontology reference.

Example 13: The device of Example 9, wherein determining external knowledge is part of a knowledge grounding process.

Example 14: The device of Example 9, wherein the criteria machine learning model is implemented with a sequence-to-sequence technique.

Example 15: The device of Example 9, wherein training the external knowledge machine learning model is further based on a criteria type of at least one of the eligibility criteria.

Example 16: A computer-readable storage medium storing instructions that, when executed by a processor, cause the processor to: receive a plurality of clinical trial protocol titles and associated eligibility criteria; train an entity machine learning model with the plurality of clinical trial protocol titles and associated eligibility criteria, wherein the trained entity machine learning model identifies an individual entity associated with an eligibility criteria that is associated with an individual brief description of an individual clinical trial; use the entity machine learning model to infer a plurality of entities associated with a plurality eligibility criteria of clinical trial protocol titles; train a criteria machine learning model with the plurality of clinical trial protocol titles and the plurality of entities as inputs and the associated eligibility criteria as outputs; using the entity machine learning model, identify an entity associated with a protocol title of a clinical trial; and using the criteria machine learning model, generate one or more eligibility criteria for a clinical trial based on the protocol title of the clinical trial and the entity.

Example 17: The computer-readable storage medium of Example 16, wherein the instructions further cause the processor to: train a category machine learning model with the plurality of clinical trial protocol titles and category labels for each of the clinical trial protocol titles; use the category machine learning model to infer a plurality of categories associated with a plurality of eligibility criteria of clinical trial protocol titles, wherein the criteria machine learning model is further trained with the plurality of categories; and using the category machine learning model, identify a category associated with the eligibility criteria of the protocol title of the clinical trial.

Example 18: The computer-readable storage medium of Example 16, wherein entity machine learning model is additionally trained based on an indication of criteria type for each of the associated eligibility criteria.

Example 19: The computer-readable storage medium of Example 18, wherein the criteria type indicates whether a criteria comprises an inclusion criteria or an exclusion criteria.

Example 20: The computer-readable storage medium of Example 16, wherein the instructions further cause the processor to: iteratively receive modifications of the protocol trial and produce corresponding updated eligibility criteria.

In closing, although the various configurations have been described in language specific to structural features and/or methodological acts, it is to be understood that the subject matter defined in the appended representations is not necessarily limited to the specific features or acts described. Rather, the specific features and acts are disclosed as example forms of implementing the claimed subject matter.

1. A method for generating eligibility criteria, comprising:
receiving a brief description of a clinical trial;
determining external knowledge derived from the brief description of the clinical trial;
using a machine learning model to infer, based on the brief description and the selected external knowledge, eligibility criteria for the clinical trial.

2. The method of claim 1, wherein the external knowledge includes a category of an eligibility criteria associated with the brief description.

3. The method of claim 2, wherein the category is inferred from a machine learning model trained on a corpus of clinical trial eligibility criteria that has been labeled with semantic categories.

4. The method of claim 1, wherein the external knowledge includes an entity associated with a portion of an eligibility criteria associated with the brief description of the clinical trial.

5. The method of claim 4, wherein the entity is identified using a machine learning model that utilizes extreme multi-label classification to associate the portion of the eligibility criteria associated with the brief description with one of a number of entities.

6. The method of claim 4, wherein the entity is selected using a sequence-to-sequence technique with the brief description of the clinical trial as an input and the entity as an output.

7. The method of claim 4, wherein entity selection is framed as an information retrieval problem wherein the brief description comprises a query and entities comprise documents that are searched.

8. The method of claim 1, wherein the brief description of the clinical trial comprises a protocol title of the clinical trial.

9. A device comprising:
one or more processors; and
a computer-readable storage medium having encoded thereon computer-executable instructions that cause the one or more processors to:
receive a plurality of clinical trial protocol titles and associated eligibility criteria;
train an external knowledge machine learning model with the plurality of clinical trial protocol titles and associated eligibility criteria, wherein the external knowledge machine learning model identifies external knowledge associated with a portion of one of the associated eligibility criteria;
use the external knowledge machine learning model to infer a plurality of pieces of external knowledge associated with a plurality of clinical trial protocol titles;
train a criteria machine learning model with the plurality of clinical trial protocol titles and the plurality of pieces of external knowledge as inputs and the associated eligibility criteria as outputs; and
using the criteria machine learning model, generate one or more eligibility criteria for a clinical trial based on a protocol title of the clinical trial.

10. The device of claim 9, wherein the instructions further cause the one or more processors to:
using the external knowledge machine learning model, identify a plurality of entities associated with eligibility criteria associated with the protocol title of the clinical trial, wherein training the criteria machine learning model is based in part on the identified plurality of entities.

11. The device of claim 9, wherein the eligibility criteria includes an inclusion criteria or an exclusion criteria.

12. The device of claim 9, wherein the instructions further cause the one or more processors to:
normalize medical terminology within the eligibility criteria associated with the protocol title using a medical ontology reference.

13. The device of claim 9, wherein determining external knowledge is part of a knowledge grounding process.

14. The device of claim 9, wherein the criteria machine learning model is implemented with a sequence-to-sequence technique.

15. The device of claim 9, wherein training the external knowledge machine learning model is further based on a criteria type of at least one of the eligibility criteria.

16. A computer-readable storage medium storing instructions that, when executed by a processor, cause the processor to:
receive a plurality of clinical trial protocol titles and associated eligibility criteria;
train an entity machine learning model with the plurality of clinical trial protocol titles and associated eligibility criteria, wherein the trained entity machine learning model identifies an individual entity associated with an eligibility criteria that is associated with an individual brief description of an individual clinical trial;
use the entity machine learning model to infer a plurality of entities associated with a plurality eligibility criteria of clinical trial protocol titles;
train a criteria machine learning model with the plurality of clinical trial protocol titles and the plurality of entities as inputs and the associated eligibility criteria as outputs;
using the entity machine learning model, identify an entity associated with a protocol title of a clinical trial; and
using the criteria machine learning model, generate one or more eligibility criteria for a clinical trial based on the protocol title of the clinical trial and the entity.

17. The computer-readable storage medium of claim 16, wherein the instructions further cause the processor to:
train a category machine learning model with the plurality of clinical trial protocol titles and category labels for each of the clinical trial protocol titles;
use the category machine learning model to infer a plurality of categories associated with a plurality of eligibility criteria of clinical trial protocol titles, wherein the criteria machine learning model is further trained with the plurality of categories; and
using the category machine learning model, identify a category associated with the eligibility criteria of the protocol title of the clinical trial.

18. The computer-readable storage medium of claim 16, wherein entity machine learning model is additionally trained based on an indication of criteria type for each of the associated eligibility criteria.

19. The computer-readable storage medium of claim 18, wherein the criteria type indicates whether a criteria comprises an inclusion criteria or an exclusion criteria.

20. The computer-readable storage medium of claim 16, wherein the instructions further cause the processor to:
iteratively receive modifications of the protocol trial and produce corresponding updated eligibility criteria.
